(19) United States
(12) Patent Application Publication
(10) Pub. No.: US 2025/0076225 A1
(43) Pub. Date: Mar. 6, 2025

(54) INFORMATION PROCESSING SYSTEM, INFORMATION PROCESSING METHOD AND PROGRAM

(71) Applicant: RIGAKU CORPORATION, Tokyo (JP)

(72) Inventors: Toshihide SHIBASAKI, Tokyo (JP); Takumi OHTA, Tokyo (JP); Akihiro HIMEDA, Tokyo (JP)

(73) Assignee: RIGAKU CORPORATION, Tokyo (JP)

(21) Appl. No.: 18/815,858

(22) Filed: Aug. 27, 2024

(30) Foreign Application Priority Data
Aug. 28, 2023 (JP) 2023-138048

(51) Int. Cl. G01N 23/20; G01N 33/38

(52) U.S. Cl. CPC G01N 23/20; G01N 33/383

(57) ABSTRACT

An information processing system is provided. The data group acquisition unit acquires a data group including a plurality of crystalline phase information. The subclass setting unit sets the crystalline phase information included in the data group into any of a first and second subclasses. A number of the first subclass(es) is one or more, a number of the second subclass(es) is zero or more, and a total number of the first and second subclasses is two or more. The crystalline phase selection unit selects the crystalline phase information from the first subclass or both of the first and second subclass, and defines the selected crystalline phase information as selected data. When there are a plurality of the first subclasses, the selected data includes the at least one crystalline phase information in each of the first subclasses. A profile generation unit generates the X-ray diffraction profile based on the selected data.

### CROSS-REFERENCE TO RELATED APPLICATION

The present application claims priority under 35 U.S.C. § 119 to Japanese Patent Application No. 2023-138048, filed Aug. 28, 2023, the contents of which are incorporated herein by reference in their entirety. The present invention relates to an information processing system, an information processing method and a program.

### BACKGROUND

### Related Art

As a method for analyzing a crystalline phase included in a powder sample (hereinafter referred to as a “qualitative analysis”), an X-ray diffraction method can be exemplified. In a qualitative analysis in a conventional X-ray diffraction method, a crystalline phase included in a powder sample can be identified by comparing a diffraction pattern generated from the powder sample with a diffraction pattern recorded in a database in advance. However, the conventional method has a problem that, since the number of crystalline phase data recorded in the database is so large, that it takes long time to collate the diffraction pattern with the information recorded in the database, and an accuracy rate of the identification of complex diffraction patterns is low.

In recent years, as a method for improving the conventional qualitative analysis, a qualitative analysis using machine learning has been examined. The qualitative analysis using the machine learning creates a neural network using training data as learning data, the training data using a diffraction pattern as input data and using a crystalline phase included in the diffraction pattern as output data. By inputting measurement result of a test sample into the neural network, a crystalline phase included in the test sample can be obtained.

Non-patent literature 1 discloses a method for creating the training data. Non-patent literature 1 describes that, when creating plural types of single-phase profiles based on the crystalline phase information recorded in the database and combining the single-phase profiles, a diffraction pattern of the mixture can be simulated, and the thus obtained diffraction pattern can be used for machine-learning.

### Prior Art Documents

### Non-Patent Document

[Non-Patent Document 1] Jan Schuetzke et al, Enhancing deep-learning training for phase identification in powder X-ray diffractograms, IUCrJ 8 diffractograms, IUCrJ 8 (2021) 408-420.

### SUMMARY

### Problems to be Solved by Invention

Such qualitative analysis using the machine-learning has a problem that a huge amount of training data is necessary to be learned to create a neural network, which takes long time. For example, in a cement database, about 70 types of crystalline phase information are recorded. The combinations of those types of the crystalline phase information is as large as about 10 billion types, and such a huge amount of training data has been necessary to be learned. Furthermore, as the number of the types of the crystalline phase information recorded in the database is increased, the number of the combinations is also increased dramatically, so that even more time is expected to be taken. Therefore, a method for reducing the learning time has been sought.

According to an aspect of the present invention, an information processing system for generating an X-ray diffraction profile is provided. This information processing system includes: a data group acquisition unit; a subclass setting unit; a crystalline phase selection unit; and a profile generation unit. The data group acquisition unit acquires a data group that includes a plurality of crystalline phase information. The subclass setting unit sets the crystalline phase information included in a data group into either of a first subclass and a second subclass. The number of the first subclass(es) is one or more, the number of the second subclass(es) is zero or more, and the total number of the first subclass(es) and the second subclass(es) is two or more. The crystalline phase selection unit selects the crystalline phase information from the first subclass or both of the first subclass and the second subclass, and defines the selected crystalline phase information as the selected data. When there are a plurality of the first subclasses, the selected data includes at least one crystalline phase information in each of the first subclasses. The profile generation unit generates the X-ray diffraction profile based on the selected data.

### BRIEF DESCRIPTION OF DRAWINGS

FIG. 1 is a diagram illustrating an example of a system configuration of an information processing system.

FIG. 2 is a diagram illustrating an example of a hardware configuration of an information processing apparatus.

FIG. 3 is a diagram illustrating an example of a functional configuration of the information processing apparatus.

FIG. 4 is a flowchart illustrating an example of information processing during learning of the information processing apparatus.

FIG. 5 is a diagram illustrating an example of a setting screen.

FIG. 6 is a diagram illustrating a modified example of the setting screen.

FIG. 7 is a flowchart illustrating an example of information processing during inference of the information processing apparatus.

FIG. 8 is a schematic diagram illustrating an example of processing for determining a crystalline phase.

FIG. 9 is a diagram illustrating a difference in learning data pattern between the cases of using and not using a subclass.

FIG. 10 is a diagram illustrating an example of comparison in performance between the cases of using and not using the subclass.

FIG. 11 is a diagram illustrating an example of comparison in learning time between the cases of using and not using the subclass.

### DETAILED DESCRIPTION

Hereinafter, embodiments of the present invention will be described with reference to drawings. The various features illustrated in the following embodiments can be combined with each other.

In the present application, the “unit” may include, for instance, a combination of hardware resources implemented by a circuit in a broad sense and information processing of software that can be concretely realized by these hardware resources. Further, various information is performed in Embodiment 1, and the information can be represented by, for instance, high and low signal values as a set of binary bits consisting of zero or one, and communication/calculation can be performed on a circuit in a broad sense.

Further, the circuit in a broad sense is a circuit realized by combining at least an appropriate number of a circuit, a circuitry, a processor, a memory, and the like. In other words, it is a circuit which includes application specific integrated circuit (ASIC), programmable logic device (e.g., simple programmable logic device (SPLD), complex programmable logic device (CPLD), and field programmable gate array (FPGA)), and the like.

### Embodiment 1

### 1. System Configuration Diagram

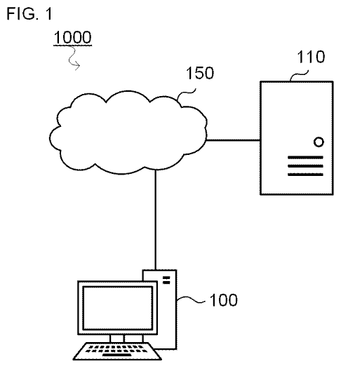

FIG. 1 is a diagram illustrating an example of a system configuration of an information processing system 1000. As illustrated in FIG. 1, the information processing system 1000 includes, as its system configuration, an information processing apparatus 100 and an X-ray diffractometer 110. The information processing apparatus 100 and the X-ray diffractometer 110 are communicably connected via a network 150. The information processing apparatus 100 is an apparatus for generating an X-ray diffraction profile, and performs processing according to Embodiment 1 including that of Modified Example described below. The X-ray diffractometer 110 is a device for measuring diffraction which is caused as a result of scatter and interference of X-rays by electrons around atoms of a sample, when the sample is irradiated with the X-rays.

Herein, the information processing system according to the claims may be composed of plural devices or a single device. When the information processing system according to the claims is composed of a single device, an example of the device is an information processing apparatus 100. When the information processing system according to the claims is composed of plural devices, examples of the plural devices include: the information processing apparatus 100 and the X-ray diffractometer 110; a cloud system that provides functions of the information processing apparatus 100; and the like.

### 2. Hardware Configuration

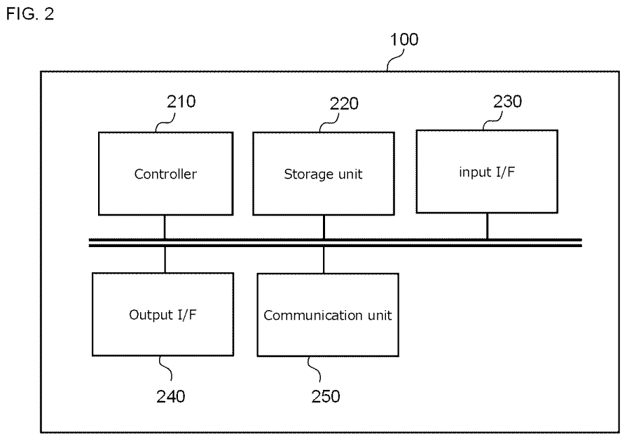

FIG. 2 is a diagram illustrating an example of a hardware configuration of the information processing apparatus 100. As illustrated in FIG. 2, the information processing apparatus 100 includes, as its hardware configuration, a controller 210, a storage unit 220, an input interface (hereinafter, referred to as an “input I/F”) 230, an output interface (hereinafter, referred to as an “output I/F”) 240 and a communication unit 250.

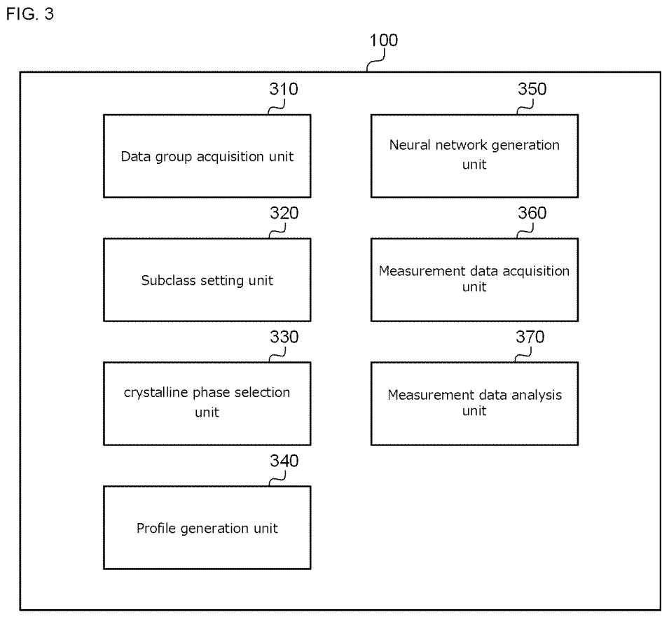

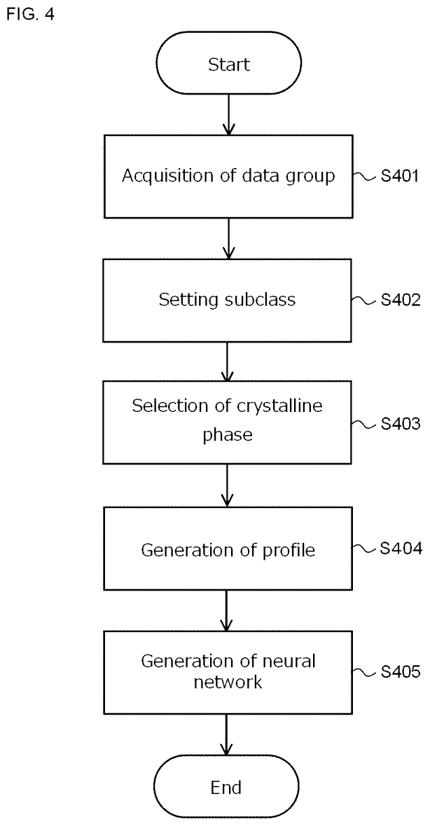

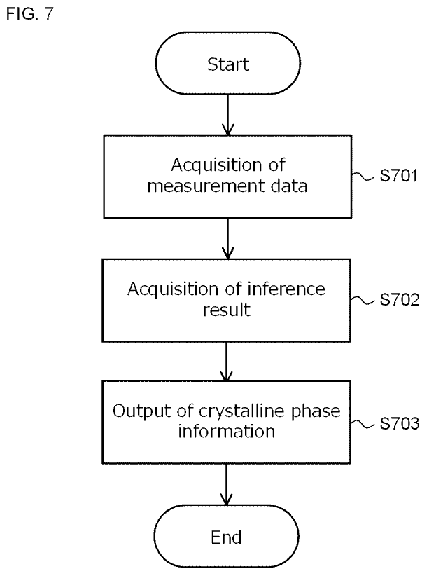

The controller 210 is a CPU (Central Processing Unit) or the like, and controls an entire of the information processing apparatus 100. The controller 210 executes processing based on a program stored in the storage unit 220, thereby realizing a functional configuration of the information processing apparatus 100 illustrated below in FIG. 3, below-described processing of flow charts illustrated in FIGS. 4 and 7 and the like.

The storage unit 220 is any of a HDD (Hard Disk Drive), a ROM (Read Only Memory), a RAM (Random Access Memory), an SSD (Solid State Drive) or any of combinations thereof, and stores program and data or the like used by the controller 210 to execute the processing based on the program. The storage unit 220 is an example of a storage medium. Incidentally, in the present specification, the data used by the controller 210 to execute the processing based on the program is assumed to be stored in the storage unit 220 for the explanation, but may be stored in a storage unit or the like of another device that can communicate with the information processing apparatus 100. That is, the data may be stored in a storage unit of any devices as long as the controller 210 can refer to the data.

The input I/F 230 is an interface that connects an input device to the information processing apparatus 100. As the input device, for example, a keyboard, a mouse and the like can be exemplified. Information that is input from the input device is delivered to the controller 210 via the input I/F 230.

The output I/F 240 is an interface that connects an output device to the information processing apparatus 100. As the output device, for example, a display can be used. Information that is output via the output I/F 240 based on the control of the controller 210 is output to the output device.

The communication unit 250 is an NIC (Network Interface Card) or the like, which connects the information processing apparatus 100 to the network 150, and administers the communication with other devices.

### 3. Functional Configuration

FIG. 3 is a diagram illustrating an example of a functional configuration of the information processing apparatus 100. As illustrated in FIG. 3, the information processing apparatus 100 includes, as its functional configuration, a data group acquisition unit 310, a subclass setting unit 320, a crystalline phase selection unit 330, a profile generation unit 340, a neural network generation unit 350, a measurement data acquisition unit 360 and a measurement data analysis unit 370.

The data group acquisition unit 310 acquires a data group that includes a plurality of crystalline phase information from the storage unit 220 and the like. Herein, the crystalline phase information denotes information that enables to calculate a diffraction pattern, and examples thereof include crystal structure information and intensity information at a certain angle (2θ). A data group is preferably a structured set of information like a database, but not limited to it. The data group is not limited to those provided, and the user may collect and record a plurality of the crystalline phase information, and use the recorded crystalline phase information as a data group.

The subclass setting unit 320 sets the crystalline phase information included in the data group into either of the first subclass and the second subclass. Herein, the number of the first subclass(es) is one or more, the number of the second subclass(es) is zero or more, and the total number of the first subclass(es) and the second subclass(es) is two or more. Herein, the crystalline phase information included in the data group is necessarily classified into either of the first subclass and the second subclass. If possibly, there is crystalline phase information that is not classified in either of the subclasses, the crystalline phase information is assumed to be classified into the second subclass. The subclass setting unit 320 may perform the setting based on an input of the user, or may perform automatically. Further, the subclass is preferably determined based on a kind of an analysis subject or a purpose of the analysis. As the kind of the analysis subject, cement and battery materials and the like can be exemplified, and as the purpose of the analysis, analysis of impurity components, determination of crystal polymorphism and the like can be exemplified.

The crystalline phase selection unit 330 selects the crystalline phase information from the first subclass or both of the first subclass and the second subclass, and defines the crystalline phase information as the selected data. Here, when there are a plurality of the first subclasses, the selected data includes at least one crystalline phase information in each of the first subclasses. By selecting the crystalline phase information as described above, the number of the combinations of the crystalline phase information included in the selected data can be reduced. For example, by setting the crystalline phase information, which is considered as a main component, into the first subclass, and setting the crystalline phase information, which is considered as an impurity, into the second subclass, the selected data which is sure to include the main component can be created. The above description does not exclude to set the crystalline phase information, which is considered as the impurity, into the first subclass, but may set the crystalline phase information, which is sure to include the impurity, into the first subclass. Incidentally, in what way the subclass is set, by setting the one or more first subclasses, the number of the combinations of the types of the crystalline phase information included in the selected data can be reduced, whereby the effect of Embodiment 1 can be obtained.

The profile generation unit 340 generates an X-ray diffraction profile based on the selected data that is selected by the crystalline phase selection unit 330. More specifically, the profile generation unit 340 preferably generates a plurality of single-phase profiles from the selected data, and then generates an X-ray diffraction profile by combining the plurality of the generated single-phase profiles. Herein, the combination of the profiles means summing up the intensity information using the plurality of the crystalline phase information and their blend ratio, so that the generated X-ray diffraction profile is the X-ray diffraction profile of the mixture. Incidentally, the above description shows a method for efficiently generating the X-ray diffraction profile of the mixture, and does not exclude to generate the X-ray diffraction profile of the mixture directly from the selected data.

The neural network generation unit 350 generates a neural network that is allowed to be trained by machine learning using training data which uses the X-ray diffraction profile generated by the profile generation unit 340 as input data, and uses the selected data as output data. The neural network trained by machine learning using (hereinafter, simply referred to as a “neural network”) is stored into the storage unit 220 and the like.

The measurement data acquisition unit 360 acquires the X-ray diffraction profile, which is the measurement data. The measurement data is generated by the X-ray diffractometer 110, is transmitted to the information processing apparatus 100, and is stored into the storage unit 220 and the like. The measurement data acquisition unit 360 acquires the measurement data from the storage unit 220 and the like.

The measurement data analysis unit 370 may output an inference result that infers the crystalline phase information included in the measurement data also by inputting the measurement data into the learned neural network. The measurement data analysis unit 370 may output the inference result either by displaying the inference result on an output device via the output I/F 240 or the like, or by writing the inference result into a file or the like and storing the inference result into the storage unit 220. Herein, the inference result is the result of inferring the crystalline phase information included in the measurement data, and is a continuous value from zero to one which is related to probability of including a certain crystalline phase.

The measurement data analysis unit 370 may input the measurement data into the neural network so as to acquire the inference result that infers the crystalline phase information included in the measurement data, thereby outputting the crystalline phase information included in the measurement data based on the inference result. As a method for determining the crystalline phase information included in the measurement data, for example, a certain threshold value may be used with respect to the inference result. The measurement data analysis unit 370 outputs at least one of the inference result and the crystalline phase information included in the measurement data.

### 4. Information Processing

### (1) During Learning

FIG. 4 is a flowchart illustrating an example of information processing during learning of the information processing apparatus 100.

In step S401, the data group acquisition unit 310 acquires a data group that includes a plurality of the crystalline phase information from the storage unit 220 and the like.

In step S402, the subclass setting unit 320 controls the output device to display a setting screen for setting the plurality of crystalline phase information included in the data group into either of the first subclass and the second subclass, based on the plurality of crystalline phase information included in the data group.

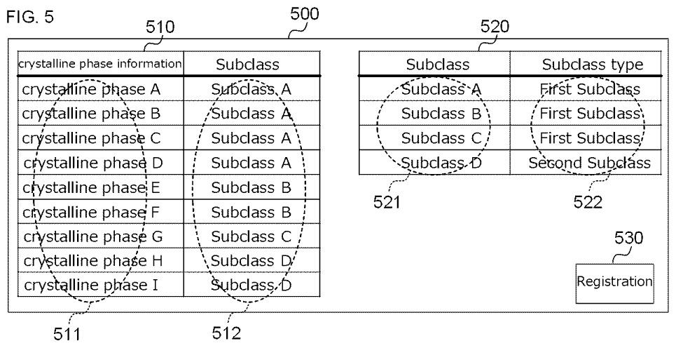

FIG. 5 is a diagram illustrating an example of the setting screen 500. The setting screen 500 includes a setting window 510 for the subclass, a setting window 520 for a subclass type and a registration button 530. Further, the setting window 510 for the subclass includes a crystalline phase information display column 511 and a subclass setting column 512, and a setting window 520 for the subclass type includes a subclass display column 521 and a subclass type setting column 522. As described above, in the setting column 522 for the subclass type, either of the first subclass and the second subclass can be set, and each of the subclasses, which are set in the subclass setting column 512, can be set whether to correspond to the first subclass or the second subclass. Setting the crystalline phase information included in the data group into the first subclass and the second subclass means setting both of the subclass and the subclass type for the crystalline phase information.

The crystalline phase information display column 511 displays the crystalline phase information included in the data group. The user may input the subclass name into the subclass setting column 512 for each of the crystalline phases, or may select the subclass name from a list box. Further, the subclasses may also be assigned automatically based on the information included in the data group, not input by the user.

The subclass display column 521 displays the subclass that is set in the subclass setting column 512. For each of the subclasses displayed in the subclass display column 521, either of the first subclass and the second subclass is selected, and is set in the subclass type setting column 522. Also in this case, the subclass type may be selected from the list box, or either of the first subclass and the second subclass may be selected from a check box. In FIG. 5, the setting window 510 for the subclass name and the setting window 520 for the subclass type are displayed on the same screen, but they may be displayed on different screens. By pressing the registration button 530, information on the setting of the subclass is updated.

Incidentally, the list box is an example of a GUI element, and any GUI element may be adopted if the subclasses can be set for the crystalline phase information. In FIG. 5, the subclass setting column 512 and the setting column 522 for the subclass type are separated, but both of the subclass and the subclass type may be set at once, like the n-th first subclass.

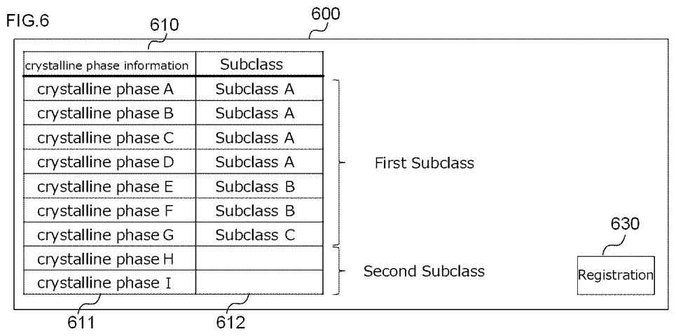

FIG. 6 illustrates a modified example of the setting screen 500. Explanation overlapped with that of FIG. 5 will be omitted. The setting screen 600 includes a setting window 610 for the subclass and a registration button 630, and the setting window 610 for the subclass includes a crystalline phase information display column 611 and a subclass setting column 612. In the subclass setting column 612, the subclass name may be listed only for the crystalline phase that corresponds to the first subclass. In this case, the crystalline phase with no subclass name listed may be treated as the second subclass.

The subclass setting unit 320 sets the crystalline phase information included in the data group into either of the first subclass and the second subclass based on an operation via the setting screen. As illustrated in FIG. 6, when the crystalline phase corresponds to none of the subclasses, the crystalline phase is treated as the second subclass, so that the subclassification is performed so as not to include the crystalline phase information that belongs to none of the subclasses.

In step S403, the crystalline phase selection unit 330 selects the crystalline phase information from the first subclass or both of the first subclass and the second subclass, and defines the crystalline phase information as the selected data. Herein, when there are a plurality of the first subclasses, the selected data includes at least one crystalline phase information in each of the first subclass.

In step S404, the profile generation unit 340 generates an X-ray diffraction profile (a mixture profile) based on the selected data selected by the crystalline phase selection unit 330.

In step S405, the neural network generation unit 350 generates a neural network that is allowed to be trained by machine learning using training data which uses the X-ray diffraction profile generated by the profile generation unit 340 as input data, and uses the selected data as output data.

### (2) During Inference

FIG. 7 is a flowchart illustrating an example of information processing during inference of the information processing apparatus 100.

In step S701, the measurement data acquisition unit 360 acquires the measurement data. The measurement data acquisition unit 360 acquires the measurement data measured by the X-ray diffractometer 110 from the storage unit 220 and the like.

In step S702, the measurement data analysis unit 370 inputs the measurement data into the learned neural network so as to acquire the inference result that infers the crystalline phase information included in the measurement data. In addition, the crystalline phase information included in the measurement data may also be acquired based on the inference result.

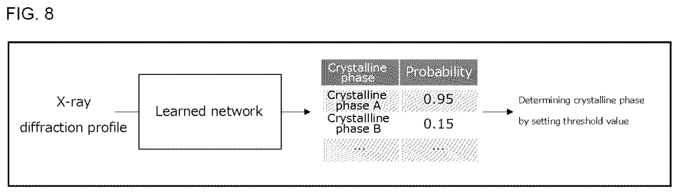

FIG. 8 is a schematic diagram illustrating an example of processing for acquiring the crystalline phase information. Likelihood that each of the crystalline phases is included in the measurement data is output as a continuous value between zero and one, and thus is described as a probability in FIG. 8 for convenience. A threshold value is set for the inference result so as to acquire the crystalline phase information included in the measurement data.

In step S703, the measurement data analysis unit 370 outputs at least one of the inference result and the crystalline phase information calculated from the inference result. The measurement data analysis unit 370 may output the inference result by displaying the inference result on the output device via an output I/F 240 or the like, or by writing the inference result into a file or the like and storing the inference result into the storage unit 220.

### 5. Embodiment

In Embodiment, explanation will be provided based on assumption that the numbers of the crystalline phase information included in the first subclass and the second subclass are two or more, respectively. Further, in Embodiment, the explanation will be provided by way of an example in which a kind of an analysis subject is cement.

When the kind of the analysis subject is cement, the subclass setting unit 320 may set a subclass to include C3S (alite), C2S (belite), C3A (aluminate) and C4AF (ferrite) as the first subclass.

In a process of producing cement, C3S, C2S, C3A and C4AF are used as raw materials. Although there are plural types of crystalline phases in C3S, C2S, C3A and C4AF respectively, at least one type of crystalline phase information is selected from each of C3S, C2S, C3A and C4AF. Therefore, it is considered that the training data can be created efficiently by setting C3S, C2S, C3A and C4AF as the first subclass and setting the impurity phase as the second subclass.

As the crystalline phase information included in C3S, C2S, C3A and C4AF, for example, following crystalline phase information are included.

### C3S

Ca₃(SiO₄) O in chemical formula, with various crystal systems such as a monoclinic system, a rhombohedral system, a hexagonal system and the like.

### C2S

Ca₂(SiO₄) in chemical formula, with various crystal systems such as a monoclinic system, a orthorhombic system, a tetragonal system, a rhombohedral system, a hexagonal system and the like.

### C3A

Ca₃(Al₂O₆) in chemical formula as a base with Na, Fe or Si added thereto in various composition ratios. As various crystal systems of C3A, an orthorhombic system, a cubic system and the like are included.

### C4AF

Ca₂FeAlO₅ in chemical formula as a base with Ca added

As a crystalline system thereof, an orthorhombic system is included.

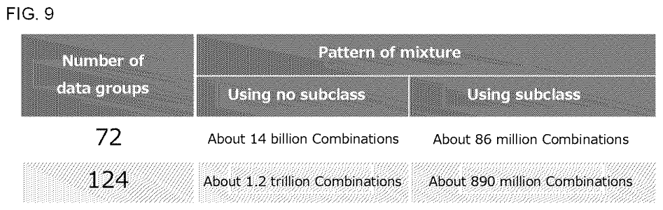

FIG. 9 is a diagram illustrating a difference in pattern of mixture combinations between the cases of using and not using the subclass. The next figure illustrates a verification result of an effect of setting the subclasses for the data groups of cement which include 72 types and 124 types of crystalline phases, respectively. Generally, cement is composed of four main components (C3S, C2S, C3A and C4AF) and a minor constituent. There are plural types of the crystalline phases in each of the main components. The example in FIG. 9 illustrates a case where the information processing apparatus 100 sets totally five subclasses, which are for the respective main components and the minor constituent. FIG. 9 is a diagram illustrating a difference in pattern of the combinations of the mixtures (eight phases) between the cases of using and not using the subclass. Thus, according to the increase of the number of the data groups, the patterns of the combinations of the mixtures is increased dramatically.

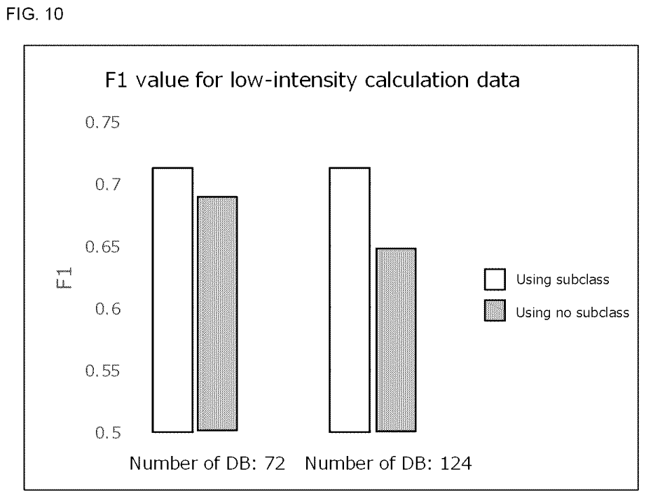

FIG. 10 is a diagram illustrating an example of comparison in performance between the cases of using and not using the subclass. In both of the cases of using and not using the subclass, the same numbers of the training data are created, and a neural network is allowed to learn. Thereafter, 1000 low-intensity cement simulation profiles are input into the learned neural network, and the crystalline phase information included in the simulation profile is inferred.

Herein, an F1 value is defined by a formula shown below.

$$Recall = TP / ( TP + FN ) \qquad [ Number 1 ]$$

$$Precision = TP / ( TP + FP )$$

$$F 1 = 2 \times \frac{Recall \times Precision}{Recall + Precision}$$

TP, FP, and FN denote true positive, false positive, and false negative, respectively. As the F1 value is closer to one, FP and FN are indicated to be small in good balance.

Similarly, in both of the cases where the numbers of the data groups are 72 and 124, the scores in the case of using the subclass are higher than the case of not using the subclass. Further, it can be resulted in that, in the case of not using the subclass, the F1 value is decreased significantly as the number of data groups is increased, on the other hand, in the case of using the subclass, even when the number of the data groups is increased, the F1 value is substantially constant. This result is considered to be obtained because the patterns of the combinations of the mixtures can be reduced by using the subclass, and indicates that the F1 value is less likely to be affected by the increase of the number of the data groups.

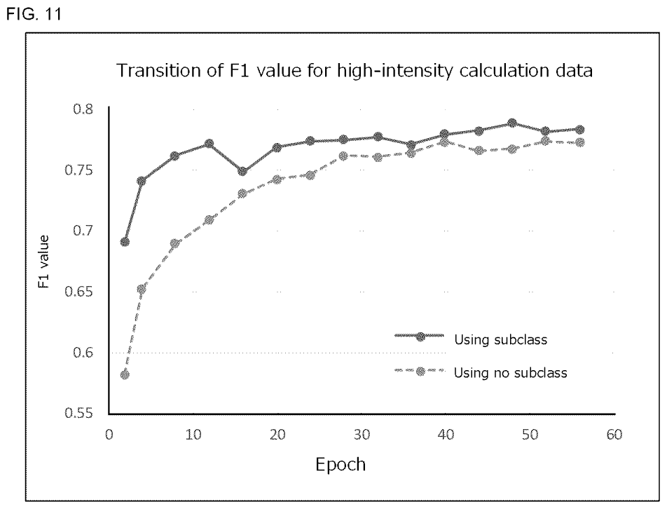

FIG. 11 is a diagram illustrating an example of comparison in learning time between the cases of using and not using the subclass. In both of the cases of using and not using the subclass, the same number of the training data is created, and the neural network is allowed to learn. Thereafter, 1000 high-intensity cement simulation profiles are input into the learned neural network, and the crystalline phase information included in the simulation profiles is inferred. FIG. 11 illustrates transitions of the F1 values during the learning process. The F1 values in the case of using the subclass reached higher scores earlier than the F1 values in the case of not using the subclass. This means that the higher performance can be achieved even in a short period of time. Incidentally, the epoch in FIG. 11 denotes a unit of learning. In the example of FIG. 11, 500 thousand data are learned per one epoch. The epoch number is in proportional to the learning time.

According to the processing of Embodiment, the patterns of the combinations of the mixtures in the learning data can be limited by setting the subclasses in the target data group.

As a result, the performance of the neural network can be improved more than that in the case of setting no subclass. In addition, the result has implied that setting of the subclass is useful when the number of the data groups is large, or when learning in a short period of the learning time.

### Modified Example 1

A modified example of Embodiment will be described below.

In Embodiment, the explanation has been provided by way of the example of cement as a kind of the analysis subject. However, the analysis subject is not limited to cement. Another example of the analysis subject is a battery material. In Modified Example 1, when the kind of the analysis subject is a battery material, the subclass setting unit 320 sets a subclass to include at least one of: an anode material; a cathode material; a solid electrolyte; and a current collector as the first subclass.

A battery is made by combining several kinds of materials such as a cathode material, an anode material, a solid electrolyte, a current collector and the like. Further, various kinds of materials are respectively used for the cathode material, the anode material, the solid electrolyte and the current collector. Therefore, by setting the cathode material, the anode material, the solid electrolyte and the current collector as the first subclass, the training data can be created efficiently.

As the crystalline phase information included in the cathode material, the anode material, the solid electrolyte and the current collector, for example, following materials are included.

### Cathode Material

Laminar rock salt-type LiCoO₂ (LCO) and LiNiCoAlO₂ (NCA); spinel-type LiMn₂O₄ (LMO); olivine-type LiFePO₄ (LFP); and sulfur-based sulfur.

### Anode Material

Carbon-based graphite; metal-based Sn and Si; oxide-based SiO; and metal Li.

### Solid Electrolyte

Sulfide-based solid electrolyte; and oxide-based solid electrolyte.

### Current Collector

Cu foil; and Al foil.

Incidentally, as a part of the cathode material and the anode material, a binder such as polyvinylidene fluoride (PVDF) and styrene butadiene rubber (SBR) may be contained.

According to Modified Example 1, the battery material can also be the analysis subject. Also in Modified Example 1, effects similar to those of Embodiment can also be achieved.

### Modified Example 2

Embodiment and Modified Example 1 have provided the example that the numbers of the crystalline phase information included in the first subclass and the second subclass are two or more, respectively, but Modified Example 2 provides an example that the number of the crystalline phase information included in the first subclass is one.

In chemical syntheses and pharmaceutical product syntheses, there are some cases that target crystalline phases are obvious. In such a case, by setting the target crystalline phase as the first subclass, and setting other compounds as the second subclass, impurity components other than the target crystalline phase can be identified.

Moreover, the present invention may be provided in each of the following aspects.

(1) An information processing system that generates an X-ray diffraction profile, comprising: a data group acquisition unit implemented by a processor configured to acquire a data group that includes a plurality of crystalline phase information; a subclass setting unit configured to set the crystalline phase information included in the data group into any of a first subclass and a second subclass, a number of the first subclass(es) being one or more, a number of the second subclass(es) being zero or more, a total number of the first subclass(es) and the second subclass(es) being two or more; a crystalline phase selection unit implemented by the processor configured to select the crystalline phase information from the first subclass or both of the first subclass and the second subclass and define the selected crystalline phase information as selected data, wherein when there are a plurality of the first subclasses, the selected data includes the at least one crystalline phase information in each of the first subclasses; and a profile generation unit implemented by the processor configured to generate the X-ray diffraction profile based on the selected data.

(2) The information processing system according to (1), wherein numbers of the crystalline phase information included in the first subclass and the second subclass are two or more, respectively.

(3) The information processing system according to (1) or (2), wherein a kind of an analysis subject is cement, and C3S (alite), C2S (belite), C3A (aluminate) and C4AF (ferrite) are included in the first subclass.

(4) The information processing system according to (1) or (2), wherein a kind of an analysis subject is a battery material, and at least one of: an anode material; a cathode material; a solid electrolyte; and a current collector is included in the first subclass.

(5) The information processing system according to any one of (1) to (4), wherein the subclass setting unit is configured to: display a setting screen, and set the crystalline phase information included in the data group into any of the first subclass and the second subclass based on an operation via the setting screen.

(6) The information processing system according to any one of (1) to (5), wherein the profile generation unit is configured to: generate a plurality of single-phase profiles from the selected data, and combine the plurality of the generated single-phase profiles so as to generate the X-ray diffraction profile.

(7) The information processing system according to any one of (1) to (6), further comprising a neural network generation unit implemented by the processor configured to generate a neural network that is allowed to be trained by machine learning using training data which uses the generated X-ray diffraction profile as input data and uses the selected data as output data.

(8) The information processing system according to (7), further comprising: a measurement data acquisition unit implemented by the processor configured to acquire the X-ray diffraction profile that is measurement data; and a measurement data analysis unit implemented by the processor configured to input the measurement data into the neural network so as to output an inference result that infers the crystalline phase information included in the measurement data.

(9) The information processing system according to (7), further comprising: a measurement data acquisition unit implemented by the processor configured to acquire the X-ray diffraction profile that is measurement data; and a measurement data analysis unit implemented by the processor configured to: input the measurement data into the neural network so as to acquire an inference result that infers the crystalline phase information included in the measurement data and output the crystalline phase information included in the measurement data based on the inference result.

(10) An information processing method executed by an information processing system that generates an X-ray diffraction profile, the method comprising: acquiring a data group that includes a plurality of crystalline phase information; setting the crystalline phase information that is included in the data group into any of a first subclass and a second subclass, number of the first subclass(es) being one or more, the number of the second subclass(es) being zero or more, the total number of the first subclass(es) and the second subclass(es) being two or more; selecting the crystalline phase information from the first subclass or both of the first subclass and the second subclass and define the selected crystalline phase information as selected data, wherein, when there are a plurality of the first subclasses, the selected data includes the at least one crystalline phase information in each of the first subclasses; and generating the X-ray diffraction profile based on the selected data.

(11) A non-transitory computer-readable memory medium storing program allowing a computer to function as the information processing system according to any one of (1) to (9).

Of course, the above aspects are not limited thereto.

Finally, various embodiments of the present invention have been described, but these are presented as examples and are not intended to limit the scope of the invention. The novel embodiment can be implemented in various other forms, and various omissions, replacements, and changes can be made without departing from the abstract of the invention. The embodiment and its modifications are included in the scope and abstract of the invention and are included in the scope of the invention described in the claims and the equivalent scope thereof.

What is claimed is:

1. An information processing system that generates an X-ray diffraction profile, comprising:
a data group acquisition unit implemented by a processor configured to acquire a data group that includes a plurality of crystalline phase information;
a subclass setting unit implemented by the processor configured to set the crystalline phase information included in the data group into any of a first subclass and a second subclass, a number of the first subclass(es) being one or more, a number of the second subclass(es) being zero or more, a total number of the first subclass(es) and the second subclass(es) being two or more;
a crystalline phase selection unit implemented by the processor configured to select the crystalline phase information from the first subclass or both of the first subclass and the second subclass and define the selected crystalline phase information as selected data, wherein when there are a plurality of the first subclasses, the selected data includes the at least one crystalline phase information in each of the first subclasses; and
a profile generation unit implemented by the processor configured to generate the X-ray diffraction profile based on the selected data.

2. The information processing system according to claim 1, wherein
numbers of the crystalline phase information included in the first subclass and the second subclass are two or more, respectively.

3. The information processing system according to claim 1, wherein
a kind of an analysis subject is cement, and
C3S (alite), C2S (belite), C3A (aluminate) and C4AF (ferrite) are included in the first subclass.

4. The information processing system according to claim 1, wherein
a kind of an analysis subject is a battery material, and
at least one of: an anode material; a cathode material; a solid electrolyte; and a current collector is included in the first subclass.

5. The information processing system according to claim 1, wherein the subclass setting unit is configured to:
display a setting screen, and
set the crystalline phase information included in the data group into any of the first subclass and the second subclass based on an operation via the setting screen.

6. The information processing system according to claim 1, wherein
the profile generation unit is configured to:
generate a plurality of single-phase profiles from the selected data, and
combine the plurality of the generated single-phase profiles so as to generate the X-ray diffraction profile.

7. The information processing system according to claim 1, further comprising a neural network generation unit implemented by the processor configured to generate a neural network that is allowed to be trained by machine learning using training data which uses the generated X-ray diffraction profile as input data and uses the selected data as output data.

8. The information processing system according to claim 7, further comprising:
a measurement data acquisition unit implemented by the processor configured to acquire the X-ray diffraction profile that is measurement data; and
a measurement data analysis unit implemented by the processor configured to input the measurement data into the neural network so as to output an inference result that infers the crystalline phase information included in the measurement data.

9. The information processing system according to claim 7, further comprising:
a measurement data acquisition unit implemented by the processor configured to acquire the X-ray diffraction profile that is measurement data; and
a measurement data analysis unit implemented by the processor configured to:
input the measurement data into the neural network so as to acquire an inference result that infers the crystalline phase information included in the measurement data and
output the crystalline phase information included in the measurement data based on the inference result.

10. An information processing method executed by an information processing system that generates an X-ray diffraction profile,
the method comprising:
acquiring a data group that includes a plurality of crystalline phase information;
setting the crystalline phase information that is included in the data group into any of a first subclass and a second subclass, a number of the first subclass(es) being one or more, the number of the second subclass(es) being zero or more, the total number of the first subclass(es) and the second subclass(es) being two or more;
selecting the crystalline phase information from the first subclass or both of the first subclass and the second subclass and define the selected crystalline phase information as selected data, wherein, when there are a plurality of the first subclasses, the selected data includes the at least one crystalline phase information in each of the first subclasses; and
generating the X-ray diffraction profile based on the selected data.

11. A non-transitory computer-readable memory medium storing program allowing a computer to function as the information processing system according to claim 1.
